(19) United States
(12) Patent Application Publication
(10) Pub. No.: US 2015/0094217 A1
(43) Pub. Date: Apr. 2, 2015

(54) TEST FOR DIAGNOSING RESISTANCE TO AZACITIDINE

(71) Applicants: UNIVERSITÉ NICE SOPHIA ANTIPOLIS, Nice (FR); CENTRE HOSPITALIER UNIVERSITAIRE DE NICE, Nice (FR)

(72) Inventors: Thomas Cluzeau, Nice (FR); Patrick Auberger, Nice (FR); Guillaume Robert, Nice (FR); Frédéric Luciano, Nice (FR)

(73) Assignees: UNIVERSITÉ NICE SOPHIA ANTIPOLIS, Nice (FR); CENTRE HOSPITALIER UNIVERSITAIRE DE NICE, Nice (FR)

(21) Appl. No.: 14/381,406

(22) PCT Filed: Feb. 28, 2013
(86) PCT No.: PCT/FR2013/000055

§ 371 Date: Aug. 27, 2014

(30) Foreign Application Priority Data
Feb. 28, 2012 (FR) 12/00584

(51) Int. Cl. G01N 33/574; C12Q 1/68

(52) U.S. Cl. CPC G01N 33/5748; C12Q 1/6886; G01N 33/57426; C12Q 2600/106; C12Q 2600/158; C12Q 2600/118; G01N 2800/52; G01N 2800/54; G01N 2333/82
USPC 506/9; 435/6.11; 435/7.1; 435/7.92

(57) ABSTRACT

The invention relates to an in vitro analysis method for predicting resistance to azacitidine treatment in a patient, using the BCL2L10 protein contained in a sample of biological fluid taken from said patient, and also biological molecules which specifically bind the BCL2L10 protein. It is characterized in that a sample of biological fluid is recovered from a patient; the percentage of total cells in said biological fluid expressing the BCL2L10 protein is calculated; this calculated percentage is compared with a reference threshold value, this threshold value being between 20% and 60%; and resistance to azacitidine treatment is diagnosed in a patient who has a percentage of cells expressing the BCL2L10 protein in said biological fluid which is greater than said reference value.

This invention relates to an analysis method enabling the in vitro diagnosis of resistance to an azacitidine treatment in a patient. The invention also relates to an in vitro analysis kit enabling the resistance of a patient to an azacitidine treatment to be predicted, as well as the use of such a kit.

Azacitidine, which has the following formula:

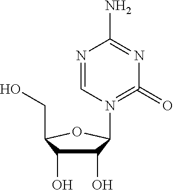

is currently the only authorized treatment for patients with myelodysplastic syndrome (MDS) and acute myeloid leukemia (AML) not eligible for hematopoietic stem cell transplant. Azacitidine is also marketed for the treatment of these diseases under the name Vidaza®.

Azacitidine (AZA) is a hypomethylating agent producing 40% to 60% response in these two diseases.

Myelodysplastic syndromes (MDS) and acute myeloid leukemia (AML) are myeloid blood diseases that develop from bone marrow stem cells, comprising precursors of the granulocyte line, corresponding to white blood cells, of the erythroblast line corresponding to red blood cells, of the megakaryocyte line corresponding to platelets and of the histio-monocyte line. MDS are characterized by significant disorders of maturation of one or all three granulocyte, erythrocyte and megakaryocyte bone marrow cell lines responsible for cytopenia. DMS can also develop into acute leukemia (AL). Traditionally, the diagnosis is based on the cytological study of the blood and the marrow, on cytogenetics and on molecular biology. DMS includes various types of anemia or refractory cytopenia as well as 5q-syndrome.

AML is characterized by the rapid proliferation of bone marrow precursors of the three granulocyte, erythrocyte and megakaryocyte lines resulting in the accumulation of immature cells in the blood and marrow, destroying normal hematopoiesis. Their diagnosis is based on the same techniques as for MDS. They include undifferentiated AML, minimally differentiated AML, myeloblastic, monoblastic, myelomonoblastic as well as acute erythroid leukemias and acute megakaryoblastic leukemias. Primary or secondary AML may be responsible for tumors in various organs or tissues (skin, ganglia, breast, digestive tract, spleen, etc.) producing myeloid sarcomas, also called chloromas or granulocytic sarcomas. They may present as acute leukemia, and present difficult diagnostic problems with malignant lymphomas.

Patients with MDS or AML treated with azacitidine are either resistant to azacitidine (“AZA resistant”), or sensitive to azacitidine (“AZA-sensitive”). However, even “AZA-sensitive” patients appear to be systematically subject to relapse after a more or less long time period has lapsed.

In other words, even if 40% of patients treated with azacitidine are immediately resistant and around 60% of patients are sensitive to the treatment during the first months, all should, in the short or medium term, develop resistance to this treatment. This phenomenon is traditionally called relapse and refers to the acquisition of resistance to a treatment to which the patient was previously sensitive.

There are currently prognostic rating systems making it possible to predict and prognosticate the overall survival of patients treated with hypomethylating agents. These systems are based on a prognostic score assessed in patient sub-groups. These are risk groups defined by the study of the karyotype and certain clinical characteristics of the patients. However, the results associated with these rating systems are unreliable response predictors.

Half of patients with MDS have a normal karyotype, and patients with identical chromosomal anomalies are often clinically heterogeneous. Somatic point mutations are common in MDS. Mutations of genes TP53, EZH2, ETV6, RUNX1 and ASXL1 are predictors of a low overall survival in patients with MDS independently of other established risk factors.

However, the current systems do not provide the appropriate results enabling the sensitivity of a patient to azacitidine to be diagnosed without administering the treatment to the patient.

At present, the only known method for determining whether a patient is resistant to an azacitidine treatment is to administer the treatment to the patient for at least 6 months and to determine whether or not the treatment has an effect.

This same method is used to identify relapse in a patient. Indeed, at present, the only way known to identify a relapse in a patient is to determine the time at which the azacitidine treatment is no longer effective for the patient. There is no method enabling the time of this relapse to be predicted before the associated symptoms appear.

When it is recommended for patients with MDS and/or AML, the azacitidine treatment is injected subcutaneously into the top of the arm, the thigh or the abdomen, daily for 7 days, and is followed by a rest period of 21 days. It may produce numerous more or less serious adverse effects such as intracranial bleeding, septicemia, change in blood pressure, lethargy, feelings of general malaise, and hair loss. In addition, the cost of an azacitidine treatment is considerable. It is around 80,000 euros per year of treatment. Today, there is no way therefore to reliably and inexpensively diagnose whether a patient is sensitive or resistant to an azacitidine treatment.

In addition, there is currently a need to predict the resistance of a patient to an azacitidine treatment in order to avoid administering azacitidine to a patient in whom this treatment is ineffective, whether it is ineffective from the start of its administration or several months later when said patient has developed resistance. In fact, administering such a treatment to a resistant patient is constraining, may be dangerous for the patient and may also lead to considerable unnecessary expenses. This applies both to the case of an azacitidine treatment recommended for patients with MDS and/or AML, and to all therapeutic azacitidine treatments. In fact, azacitidine resistance is associated with the azacitidine molecule, not the way in which it is administered.

The ability to more quickly identify patients who are immediately resistant, as well as the time of relapse of patients who are initially sensitive, is also advantageous because it makes it possible to offer other clinical tests before the clinical conditions of said patients worsen.

It is therefore essential to be capable of diagnosing, as early as possible, whether a patient will be sensitive to an azacitidine treatment and to be capable of predicting the time of the patient's relapse.

Surprisingly, the applicant was able to demonstrate a link between the expression level of the BCL2L10 protein in a biological fluid sample taken from a patient and the sensitivity of this patient to an azacitidine treatment.

Throughout the description, the general term BCL2L10 is defined as corresponding to the BCL2L10 gene, the BCL2L10 RNA transcript or the BCL2L10 protein.

The BCL2L10 gene is a member of the Bcl-2 family, which has an antiapoptotic effect in vitro. The BCL2L10 Protein shares, with the Bcl-2 protein family, the BH1, BH4 and BH2 domains. The BH3 domain, which is characteristic of proapoptotic factors of the Bcl-2 family is absent from the BCL2L10 protein. However, there are still contradictory results in the literature with regard to the proapoptotic or antiapoptotic properties of BCL2L10, in particular because its assumed ortholog in the mouse was also described as having a proapopootic activity.

BCL2L10 may interact with members of the Bcl-2 family, in particular Bcl-2, Bcl-xL and Bax in order to regulate the apoptosis in different contexts. Certain publications such as, for example, the article “Loss of BCL2L10 protein expression as prognostic predictor for poor clinical outcome in gastric carcinoma, Histopathology 2010, 57, 814-82” present BCL2L10 as an antiapoptotic gene.

The overexpression of BCL2L10 has been described as suppressing apoptosis by inhibiting cytochrome C release by the mitochondria.

Recently, it was demonstrated that the hypomethylating agent decitabine triggers apoptosis and the positive regulation, also called. “up-regulation”, of numerous genes including BCL2L10. Today, there is a link between the resistance of patients to certain anticancer treatments and the expression of the BCL2L10 gene has been demonstrated, in particular in patent application JP2010162031 (A), which describes the fact that the amplification of the BCL2L10 gene can enable cancer cells resistant to a camptothecin-based treatment to be detected. Similarly, the patent application US2009143236 (A1) describes a method for detecting the acquisition of resistance to certain drugs by the study of the amplification of certain genes, including, in particular BCL2L10. However, in these two patent applications, the anticancer agents for which resistance is evaluated do not concern azacitidine.

Patent application US2011/0129833 indicates that an increase in the expression of genes of the Bcl-2 family in a patient is correlated with a reduced likelihood that the patient will respond to a chemotherapy treatment. However, nothing in this patent application suggests that such conclusions are applicable to an azacitidine-based treatment.

The article “Role of BCL2L10 methylation and TET2 mutations in higher risk myelodysplastic, Leukemia. 2011 December; 25 (12)” describes the phenomenon of azacitidine resistance. This document describes the existence of a link between methylation of the BCL2L10 gene promoter and azacitidine resistance. Specifically, hypermethylation of the BCL2L10 gene promoter is correlated with a low survival of patients suffering from gastric cancers. This publication teaches that patients with a high methylation of the BCL2L10 promoter have a high risk of MDS and a low chance of response to epigenetic treatments such as azacitidine treatments. However, nothing in this publication teaches that a high level of expression of the BCL2L10 protein enables the same conclusion to be reached.

Instead, the publication suggests that it is a low level of expression of BCL2L10 that is correlated with a low chance of response to azacitidine. In addition, even if a high level of methylation of the BCL2L10 were truly associated with resistance to the azacitidine treatment, this data could not have been correlated with the expression level of the BCL2L10 protein. In fact, the methylation level of a gene is not necessarily associated with the expression of the protein resulting from said gene. This is in particular the case for BCL2L10.

In addition, in view of the prior art described above, nothing suggests the existence of a link between the expression level of the BCL2L10 protein and the phenomenon of azacitidine resistance. And, even more, nothing suggests the existence of a link between a high expression level of the BCL2L10 protein and azacitidine resistance.

The solution to the problem in question concerns an in vitro analysis method making it possible to diagnose resistance to an azacitidine treatment in a patient, using the BCL2L10 protein contained in a biological fluid sample taken from said patient as well as biological molecules specifically binding the BCL2L10 protein, characterized in that
a biological fluid sample is taken from a patient;
the percentage of total cells of said biological fluid expressing the BCL2L10 protein is calculated;
said calculated percentage is compared with a reference threshold value, said threshold value being between 20 and 60%; and
the resistance to an azacitidine treatment in a patient having a higher percentage of cells expressing the BCL2L10 protein in said biological fluid than said reference value is diagnosed.

Surprisingly, the applicant demonstrated the existence of a link between the percentage of cells of a biological fluid of a patient that express the BCL2L10 protein and azacitidine resistance.

The determination of a reference threshold value for this percentage, beyond which it can be concluded that a patient is resistant to azacitidine, had never before been suggested.

This method makes it possible to diagnose azacitidine resistance in a patient before said azacitidine molecule has even been administered in the patient. This method also makes it possible, advantageously, to predict the relapse of a patient who was previously sensitive to the azacitidine treatment.

The analysis method according to the invention makes it possible to avoid any unnecessary treatment of a patient with azacitidine. This is therefore advantageous in terms of health and appropriate treatment of patients, well as from an economic perspective. A second object of the invention concerns a kit for in vitro analysis enabling the in vitro analysis method according to the invention to be performed, said kit including biological molecules specifically binding the BCL2L10 protein in cells obtained from biological fluids taken from patients.

Finally, a third object of the invention concerns the use of an in vitro analysis kit according to the invention, for implementation of a method for monitoring an azacitidine treatment in order to predict relapse.

To better understand the mechanisms associated with azacitidine resistance in vitro, the inventors generated azacitidine-resistant SKM1 myeloid cells, called. AZA-R or SKM1-R. By contrast, AZA-S or SKM1-S are azacitidine-sensitive cells.

The invention will be better understood upon reading the following non-limiting description, drafted in view of the appended figures, wherein:

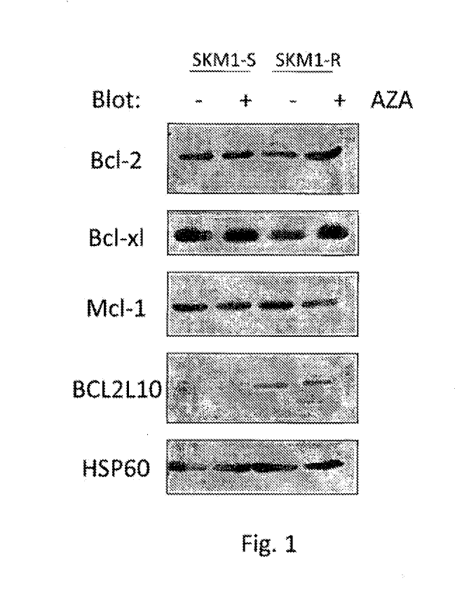

FIG. 1 shows the results of a screening of the cells obtained from SKM1 cell lines, expressing the Bcl-2 protein. The SKM1-S and SKM1-R cells are treated with 1 μM of azacitidine for 24 h. Western blot experiments are then performed in order to evaluate the amounts of Bcl-2, Mcl-1, Bcl-x1 and BCL2L10 proteins. An anti-USP60 antibody was used as a load control.

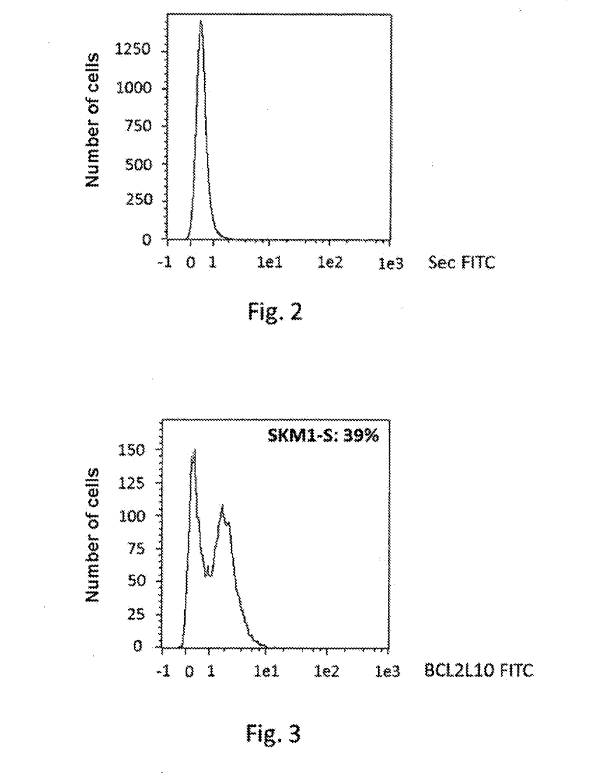

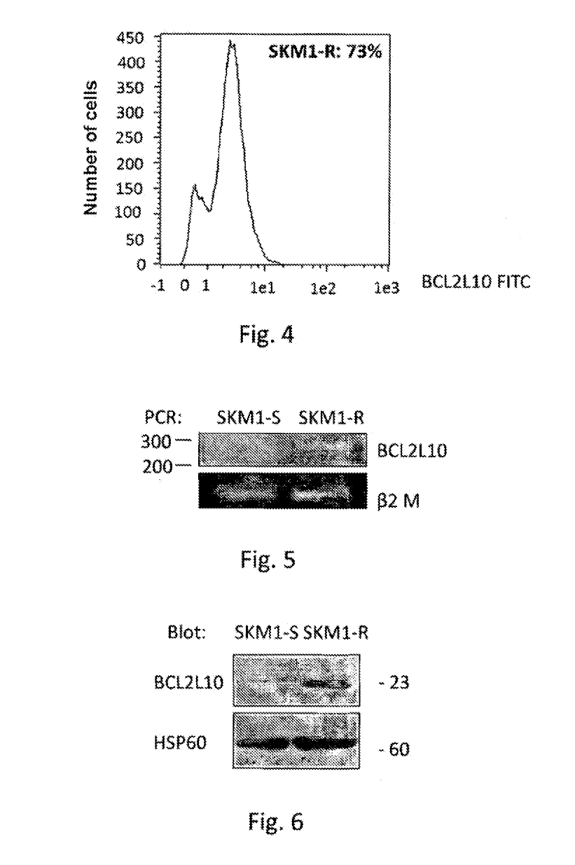

FIGS. 2 to 6 show the expression of the BCL2L10 protein in SKM1-S and SKM1-R cell lines with AML.

In FIGS. 2, 3 and 4, the BCL2L10 protein level is quantified in the SKM1-S and SKM1-R cells by flow cytometry.

FIG. 5 shows an analysis by reverse transcriptase polymerization chain reaction, referred to as RT-PCR, of the mRNA of the SKM1-S and SKM1-R cells.

FIG. 6 shows the results of a western blot enabling the protein level of BCL2L10 in SKM1-S and SKM1-R cells to be seen.

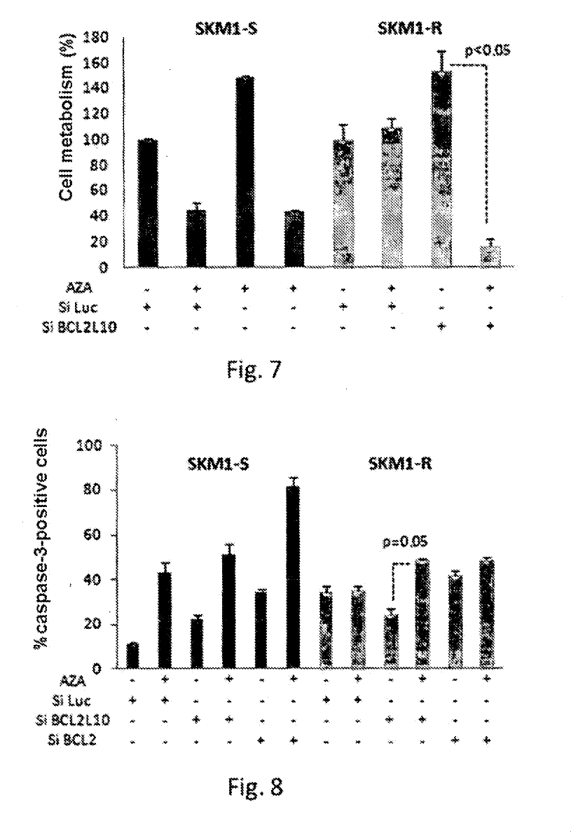

FIGS. 7 to 10 show the re-sensitization of SKM1-R cells to azacitidine followed by the extinction of the expression of the BCL2L10 gene. The extinguishing of the expression of the BCL2L10 gene is commonly referred to as “knockdown”, in this case BCL2L10 knockdown.

The SKM1-S and SKM1-R cells are transfected with an interfering RNA:Luc siRNA, BCL2L10 siRNA or Bcl-2 siRNA. After 72 h of transfection, the cells are stimulated with 1 μM of azacitidine.

In FIG. 7, the cell metabolism is measured 24 hours after stimulation by means of the XTT test (Xylose Tolerance Test). The results shown correspond to the average standard error of the mean (±SEM) of three independent experiments performed four times.

FIG. 8 shows the results of a caspase-3 labeling seen by flow cytometry 24 hours after the addition of 1 μM of azacitidine.

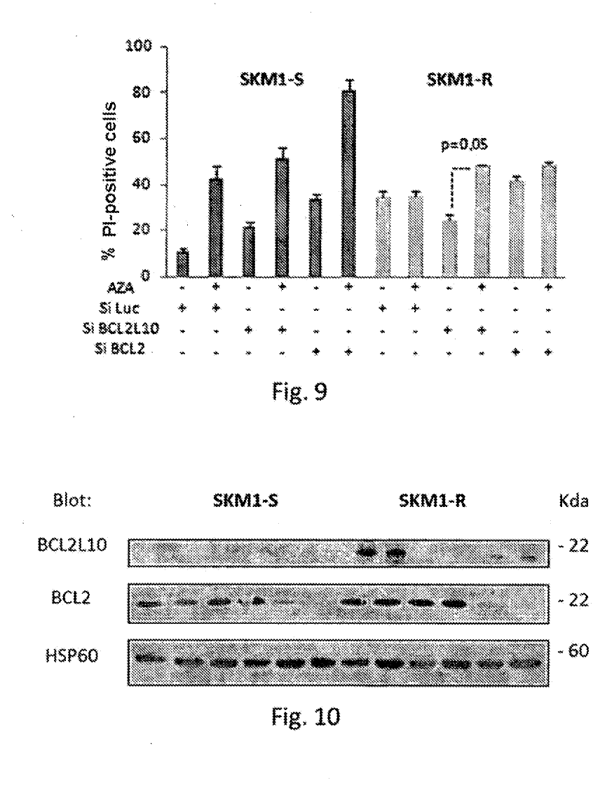

FIG. 9 shows the results of a propidium iodide (PI) labeling by flow cytometry, 24 hours after the addition of 1 μM of azacitidine.

FIG. 10 shows the results of western blots performed 24 hours after the addition of 1 μM of azacitidine in order to determine the inhibition of the expression of BCL2L10 and Bcl-2.

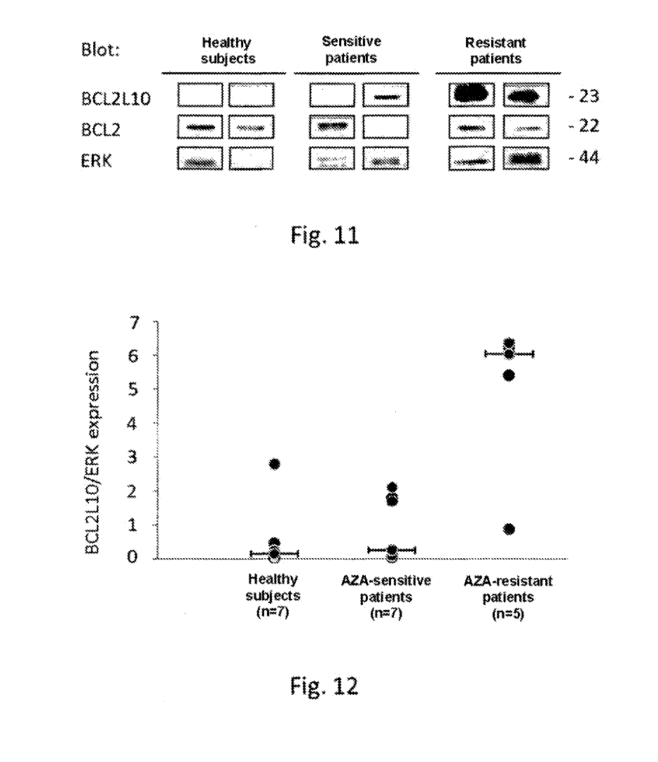

In FIGS. 11, 12 and 13, it is shown that the protein expression of BCL2L10 is specifically increased in azacitidine-resistant patients.

FIG. 11 shows the expression of BCL2L10, Bcl-2 and ERK proteins detected by western blot on “fresh” bone marrow samples from 7 healthy patients, 7 azacitidine-sensitive patients and 5 azacitidine-resistant patients. The results of the western blot are shown for two patients in each sub-croup.

FIG. 12 shows the expression of BCL2L10 and ERK proteins analyzed by means of the ImageJ software program (ImageJ is a free software program for image processing written in Java by the National Institute of Health (NIH)), and the quantification of the ratio of the expression of BCL2L10 with respect to the expression of ERK.

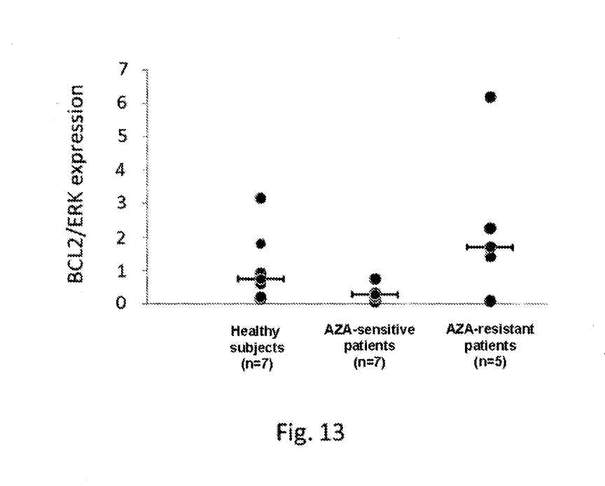

FIG. 13 shows the quantification of the expression of BCL2L10 and ERK proteins analyzed by means of the ImageJ software program and the quantification of the ratio of the expression of BCL2L10 with respect to the expression of ERK.

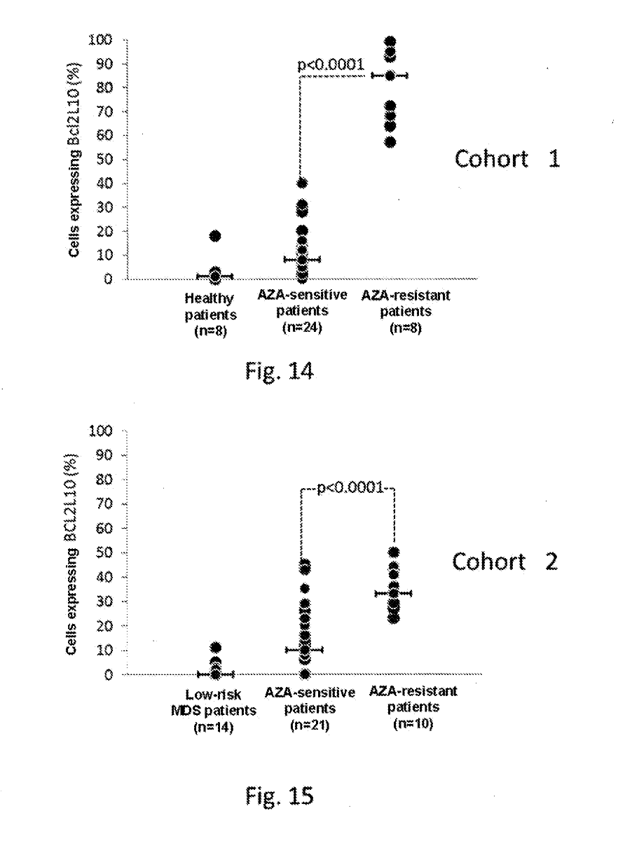

FIGS. 14 to 17 show the fact that in azacitidine-resistant patients with MDS or AML, the percentage of cells expressing the BCL2L10 protein in the bone marrow is increased.

In FIG. 14, the percentage of cells expressing the BCL2L10 protein is quantified by flow cytometry in 32 patients with MDS or AML and undergoing azacitidine treatment and in 8 healthy patients, all from cohort 1.

In FIG. 15, the percentage of cells expressing the BCL2L10 protein is quantified by flow cytometry in samples frozen in DMSO from 14 patients with low-risk MDS, 31 patients with high-risk MDS or AML and treated with azacitidine treatment, all from cohort 2.

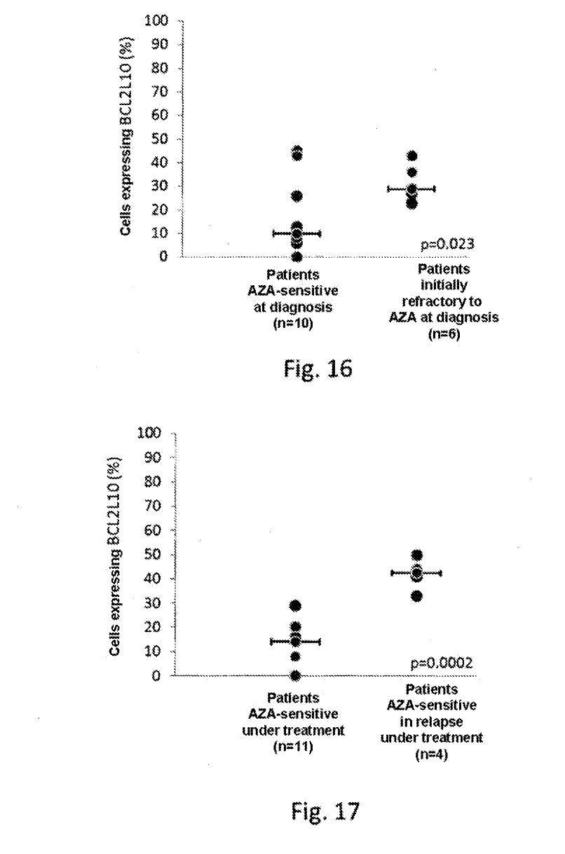

The percentage of cells expressing the BCL2L10 protein is quantified by flow cytometry in samples frozen in DMSO from 16 patients with high-risk MDS, or patients at diagnosis, as shown in FIG. 16.

The percentage of cells expressing the BCL2L10 protein is quantified by flow cytometry in samples frozen in DMSO from 15 patients with high-risk MDS or AML, all undergoing azacitidine treatment, as shown in FIG. 17.

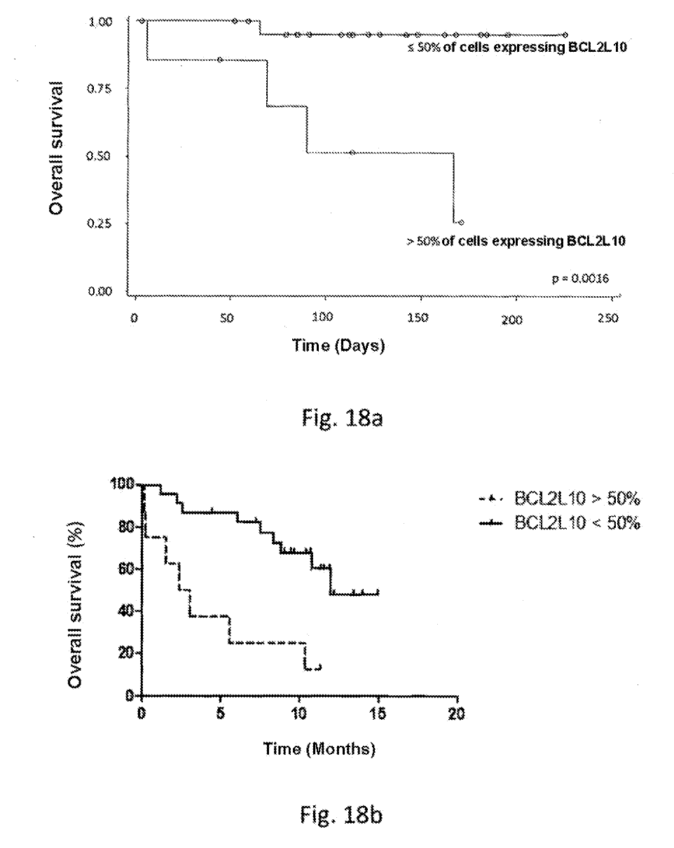

FIGS. 18a and 18b show the correlation between the percentage of cells expressing the BCL21L10 protein and the overall survival of treated patients with MDS or AML.

The comparison of the overall survival with transplantation according to Kaplan-Meier was examined for patients with MDS or AML treated with AZA, as well as the percentage of cells expressing BCL2L10 in their bone marrow.

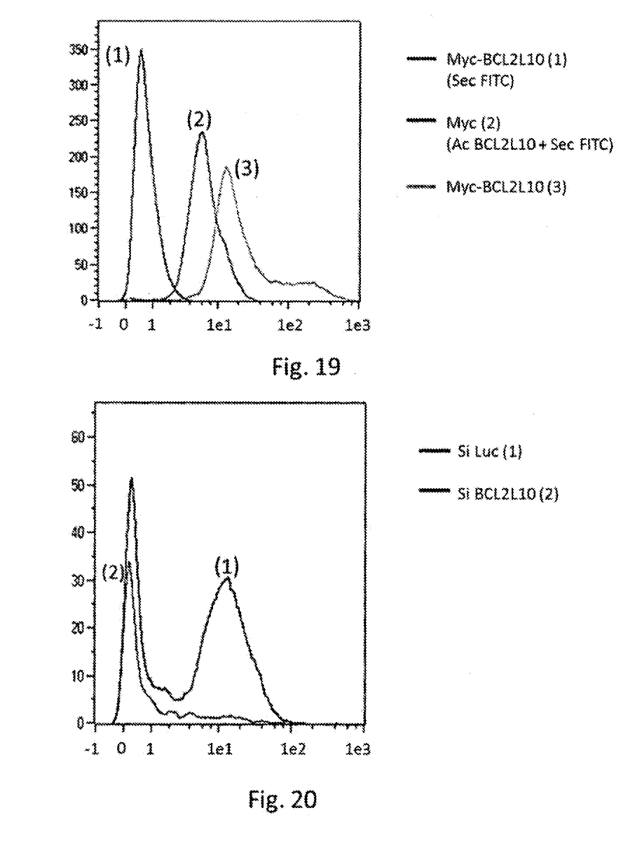

FIGS. 19 to 22 make it possible to validate the BCL2L10 protein quantification technique by flow cytometry.

In FIG. 19, cells from an HEK293 line were transfected either with pcDNA3 expression plasmids integrating the N-terminal portion of the Myc epitope tag of BCL2L10, or with pcDNA3 expression plasmids integrating the N-terminal portion of the Myc epitope tag alone. The BCL2L10 protein expression level was quantified by flow cytometry. The results of this experiment are shown in FIG. 19.

In FIG. 20, cells from an HEK293 line were transfected either with an interfering siLuc RNA or with an interfering si-BCL2L10 RNA. The BCL2L10 protein expression level was quantified by flow cytometry. The results of this experiment are shown in FIG. 20.

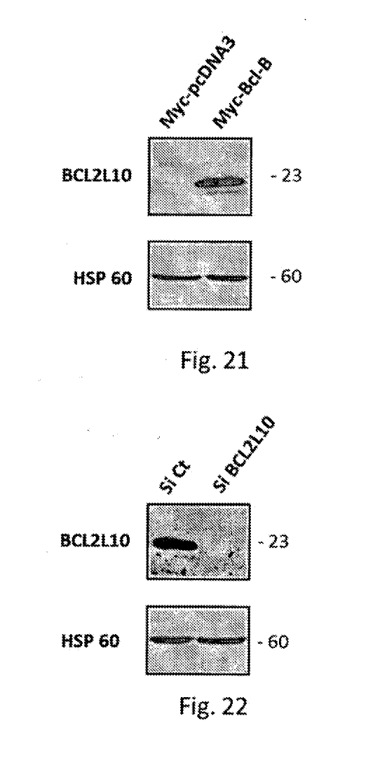

FIGS. 21 and 22 show the BCL2L10 protein level detected by western blot. An anti-HSP60 antibody is used a load control.

This invention relates to an analysis method enabling in vitro diagnosis of resistance to an azacitidine treatment in patients by performing in particular a quantification of the BCL2L10 protein expression by the total cells of a biological fluid.

According to the invention, the patients are human beings. Advantageously, the analysis method is specifically suitable for patients with malignant blood diseases such as myeloid blood diseases. Still more specifically, the patients have AML or MDS.

According to the invention, the biological fluid is a fluid obtained from the human body. As a non-limiting example of a biological fluid, mention may be made of bone marrow, blood, cerebrospinal fluid and urine. Preferably, the biological fluid according to the invention is bone marrow.

According to the invention, the term “total cells” covers all cells present in the biological fluid collected. If the biological fluid collected is bone marrow, the total cells include in particular hematopoietic stem cells (MSC) and cells of the bone marrow stroma, which are hematopoietic cells.

According to the invention, the biological molecules specifically binding the BCL2L10 protein are molecules capable of specifically binding the BCL2L10 protein. Advantageously, these are monoclonal or polyclonal antibodies, soluble receptors or aptamers, preferably monoclonal or polyclonal antibodies. Also preferably, the biological molecules specifically binding the BCL2L10 protein are monoclonal antibodies. As a non-limiting example of biological molecules specifically binding the BCL2L10 protein, mention may be made of the anti-BCL2L10 protein referenced “#3869” by the “Cell Signaling Technologies” company.

According to the invention, the reference threshold value, also called the “cut-off” value, corresponds to a percentage of BCL2L10-positive cells, i.e. a percentage of cells expressing the BCL2L10 protein, in a biological fluid.

When the percentage value of cells expressing the BCL2L10 protein in the total cells of a biological fluid obtained is greater than this “cut-off” value, the tested patients will be diagnosed as being resistant to azacitidine. Conversely, when the value obtained is below this “cut-off” value, the tested patients will be diagnosed as being sensitive to azacitidine.

According to the invention, the reference threshold value is between 20 and 60%, preferably between 30 and 55%, and more preferably, this reference threshold value is equal to 50%.

The analysis method according to the invention makes it possible to diagnose resistance to an azacitidine treatment in a patient. Said patients treated with azacitidine must generally undergo bone marrow aspirations every 1, 3 and 6 months during their treatment, then every 3 months afterward. Thus, this bone marrow sample taken as part of a traditional monitoring of azacitidine treatment may also be used for the analysis method according to the invention. It is therefore not necessarily essential to perform specific bone marrow aspirations in the patients, in view of this diagnosis of resistance to an azacitidine treatment.

In other words, it is an advantage for the patient, who will not therefore have to undergo an additional examination given that the in vitro analysis method enabling diagnosis of the patient's resistance to azacitidine will be performed on the bone marrow sample aspirated in the context of the traditional treatment.

According to the invention, the measurement of the percentage of cells of the biological fluid expressing the BCL2L10 protein is performed by flow cytometry (immunophenotyping), by hydrophobic interaction chromatography (HIC), or by quantitative polymerase chain reaction (qPCR). Preferably, this measurement is performed by flow cytometry (immunophonotyping).

The invention also relates to an in vitro analysis method enabling a patient's resistance to an azacitidine treatment to be diagnosed, by detecting the overexpression of the BCL2L10 gene contained in a biological fluid sample taken from said patient, characterized in that:
a biological fluid sample is taken from a patient;
the percentage of total cells of said biological fluid expressing BCL2L10 by detection of the overexpression of the BCL2L10 gene is calculated;
said calculated percentage is compared with a reference threshold, said threshold value being between 20 and 60%; and
the resistance to an azacitidine treatment in a patient having a percentage of cells expressing BCL2L10 in said biological fluid greater than said reference threshold value is diagnosed.

Preferentially, the detection of the overexpression of the BCL2L10 gene is performed by the comparative genomic hybridization CGH method, the flow cytometry method, the ELISA method, by the DNA chip method, or by quantitative polymerization chain reaction (qPCR). More preferentially, the detection of the overexpression of the BCL2L10 gene is performed by the comparative genomic hybridization CGH method, by the DNA chip method or by quantitative polymerization chain reaction (qPCR). Still more preferably, the detection of the overexpression of the BCL2L10 gene is performed by the DNA chip method or by quantitative polymerization chain reaction (qPCR).

The invention also relates to an in vitro analysis kit comprising biological molecules specifically binding the BCL2L10 protein in cells from a biological fluid sample taken from a patient, said kit making it possible to predict resistance to an azacitidine treatment in a patient having a percentage of cells, in said biological fluid expressing the BCL2L10 protein, greater than a reference threshold value of between 20 and 60%.

It also relates to an in vitro analysis kit comprising at least one reagent selected from the group consisting of:
a pair of primers capable of amplifying a BCL2L10 fragment, and
a probe capable of detecting the presence of BCL2L10,

said kit making it possible to predict the resistance to an azacitidine treatment in a patient having a percentage of cells, in said biological fluid expressing BCL2L10, greater than a reference threshold value of between 20 and 60%.

Another object of the invention concerns the use of a kit or of the method according to the invention, for implementing a method for monitoring an azacitidine treatment in order to predict relapse.

According to a particular embodiment of the invention, the use of the kit or the method according to the invention also makes it possible to adapt the treatment on the basis of the patients response.

According to a particular embodiment of the invention, when resistance to an azacitidine treatment in a patient is diagnosed, in particular in a patient with myelodysplastic syndrome and/or acute myeloid leukemia, an alternative treatment including at least one antitumor agent and/or anti-inflammatory agent is also administered to said patient.

Preferably, an antitumor compound chosen from alkylating agents, anti-metabolites, vegetable alkaloids, topoisomerase inhibitors, and antitumor antibiotics is administered to said patient.

As a non-limiting example of an antitumor agent that can be used according to the invention, mention may be made in particular of acadesine, also called AICAR for 5-aminoimidazole-4-carboxamide-1-β-D-ribofuranoside, derivatives of acadesine, actinomycin D, amsacrine, anthracyclines such as doxorubicin or daunorubicin, aracytin, ATRA (all-trans retinoic acid), bleomycin, bortezomib, busulfan, derivatives of camptothecin, cisplatin, carboplatin, chlorambucil, decitabine, depakine, docetaxel, derivatives of epipodophyllotoxin, erlotinib, etoposide, 5-fluorouracil (5FU), fludarabine, hydrea, ifosfamide, histone deacetylase (HDAC) inhibitors, lenalidomide, methotrexate, mitomycin C, paclitaxel, plicamycin, purinethol, thiotepa, vincristine, vinblastine and vinorelbine. Mention may also be made of the tyrosine kinase inhibitors (TKI) used in different tumor pathologies such as, for example, Imatinib, Dasatinib, Nilotinib and Sunitinib.

The derivatives of acadesine that can be used preferably have the following general formula:

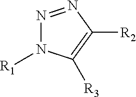

wherein
R₁ is chosen from
   a cyclic pentose group in furan form with OH groups that are free or optionally substituted with one or more mono-, bi- or triphosphate groups (or prodrugs thereof), acetyl, isopropylidene, benzoyl or para-toluoyl,
   a hexose group in pyran form with OH groups that are free or optionally substituted with one or a plurality of mono-, bi- or triphosphate groups (or prodrugs thereof) or acetyl,
   a naphthyl group, optionally substituted with one or more substituted alkyl or amino groups having 1 to 4 carbon atoms,
   a benzyl group optionally substituted with one or more substituted alkyl or amino groups having 1 to 4 carbon atoms,
   phenyl, biphenyl and heteroaryl groups;
R₂ is chosen from:
   an amide group —CONH₂, —CONHMe, —CONHEt, —CON(Me)₂, —CON(Et)₂;
   an acid or ester group —CO₂H, CO₂Me, CO₂Et, a cyano or an amidine group —CN, —C(NH₂)NH, —C(NHMe)NH, —C(NHEt)NH,
   a phenyl group optionally substituted with a halogen chosen from Cl, Br, I and F,
   a thiophene group,
   a linear or branched carbon chain having 3 to 10 carbon atoms, or
   a methoxynaphthalene group; and
R₃ is chosen from:
   a halogen group,
   a furan or —CO-furan group,
   a thiophene or —CO-thiophene or —C≡C-thiophene group,
   a toluoyl group,
   an acetylene group,
   a —CO—(CH₂)ₙ—CH₃ group, with n being between 2 and 9,
   a phenyl or —C≡C-phenyl group, optionally substituted by a halogen,
   a —C≡C—CO₂Me, —C≡C—CO₂Et, —C≡C—CONH₂ group,
   a —C≡C—(CH₂)₆CH₃ group, or
   a —C≡C-2-methoxynaphthalene group;

the racemates, enantiomers, and diastereoisomers thereof, and mixtures thereof, the tautomers thereof and the pharmaceutically acceptable salts thereof.

More preferentially, the acadesine derivatives are compounds with the following general formula;

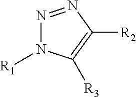

wherein R1 is

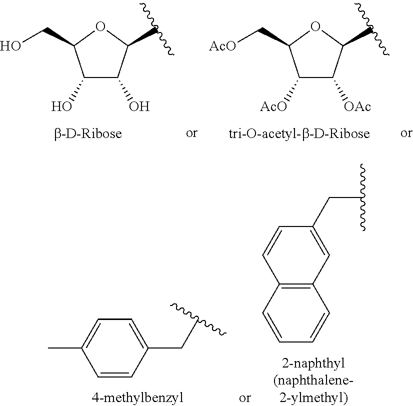

and
when R1 is a β-D-ribose group, then:
   R2=CONH₂ and R3=Cl, CO-furan, CO-thiophene or toluoyl;

or
R2=CO₂Me and R3=I or acetylene;

or
R2=phenyl and R3=I;
when R1 is a tri-O-acetyl-β-D-ribose group, then:
   R2=CO₂Et and R3=CO—(CH₂)₅—CH₃, CO-furan, toluoyl, thiophene or phenyl;

or
R2=phenyl and R3=—C≡C-phenyl;

or
R2=thiophene and R3=—C≡C thiophene;

or
R2=(CH₂)₆CH₃ and R3=—C≡C—(CH₂)₆CH₃;

or
R2=p-fluorophenyl and R3=—C≡C-p-fluorophenyl;

or
R2=2-methoxynaphthalene and

R3=—C≡C-2-methoxynaphthalene;
when R1 is a 4-methylbenzyl group, then:
   R2=CO₂Et and R3=—C≡C—CO₂Et;

or
R2=phenyl and R3=phenyl;
when R1 is a 2-naphthyl (naphthalene-2-yl-methyl) group, then:
   R2=CO₂Et and R3=I;

or
R2=CO₂Et and R3=—C≡C—CO₂Et;

or
R2=Phenyl and R3=—C≡C-phenyl;

the racemates, enantiomers and diastereoisomers thereof and mixtures thereof, the tautomers thereof and the pharmaceutically acceptable salts thereof.

As non-limiting examples of acadesine derivatives that can be used, mention may be made of the following compounds:
1′-(4-ethoxycarbonyl-5-iodo-[1,2,3]-triazol-1-yl)-2′,3′,5′-tri-O-acetyl-β-D-ribofuranose;
1′-(4-carbamoyl-5-iodo-[1,2,3]-triazol-1-yl)-β-D-ribofuranose;
1′-(4-methoxycarbonyl-5-ethynyl-[1,2,3]-triazol-1-yl)-β-D-ribofuranose;
1-(naphthyl-2-methyl)-4-ethoxycarbonyl-5-iodo-1,2,3-triazole;
1-(naphthyl-2-methyl)-4-ethoxycarbonyl-5-ethylpropiolate-1,2,3-triazole;
1′-(4-ethoxycarbonyl-5-ethylpropiolate-[1,2,3]-triazol-1-yl)-2′,3′,5′-tri-O-acetyl-β-D-ribofuranose;
1′-(4-ethoxycarbonyl-5-(2-thienyl)-[1,2,3]-triazol-1 yl)-2′,3′,5′-tri-O-acetyl-β0D-ribofuranose;
1′-(4-ethoxycarbonyl-5-phenyl-[1,2,3]-triazol-1-yl)-2′,3′,5′-tri-O-acetyl-β-D-ribofuranose;
1-(4-methylbenzyl)-4-ethoxycarbonyl-5-ethylpropiolate-1,2,3-triazole;
1′-(4-heptyl-5-(non-1-yn-1-yl)-[1,2,3]-triazol-1-yl)-2′,3′,5′-tri-O-acetyl-β-D-ribofuranose;
1′-(4-ethoxycarbonyl-5-ethylpropiolate-[1,2,3]-triazol-1-yl)-2′,3′,5′-tri-O-benzoyl-β-L-ribofuranose;
2′-deoxy-1′-(4-ethoxycarbonyl-5-ethylpropiolate-[1,2,3]-triazol-1-yl)-3′,5′-di-O-(p-toluoyl)-α-D-ribofuranose;
1′-(4-ethoxycarbonyl-5-ethylpropiolate-[1,2,3]-triazol-1-yl)-2′,3′,4′,6′-tetra-O-acetyl-β-O-glucopyranose;
1′-(4-ethoxycarbonyl-5-ethylpropiolate-[1,2,3]-triazol-1-yl)-2′,3′-O-isopropylidene-β-D-ribofuranose;
1′-(4-ethoxycarbonyl-5-ethylpropiolate-[1,2,3]-triazol-1-yl)-2′,3′-O-isopropylidene-5′-O-acetyl-β-D-ribofuranose;
1′-(4-ethoxycarbonyl-5-(2-thienyl)-[1,2,3]-triazol-1-yl)-2′,3′-O-isopropylidene-β-D-ribofuranose; and
1′-(4-ethoxycarbonyl-5-(2-thienyl)-[1,2,3]-triazol-1-yl)-2′,3′-O-isopropylidene-5′-O-acetyl-β-D-ribofuranose.

Studies were conducted to demonstrate certain advantages of this invention. The results of these studies are provided in the examples below:

### EXAMPLE 1

### Validation of the Cytometry Technique for the Detection of BCL2L10

SKM1 cells resistant to azacitidine (AZA), referred to as “SKM1-R”, defective both for apoptosis and autophagy processes, were produced. By comparison with their AZA-sensitive homologs, referred to as “SKM1-S”, the SKM1-R cells show an increased expression of the BCL2L10 protein (Bcl-B), an anti-apoptotic member of the Bcl-2 family, but the SKM1-R and SKM1-S cells show equivalent levels of Bcl-2, Bcl-xL and Mcl-1 proteins, as illustrated in FIG. 1.

An increase in the expression of BCL2L10 proteins was also found in the mass of SKM1-R cells before limited dilution, indicating that the overexpression of BCL2L10 is linked to azacitidine (AZA) resistance and is not due to a clonal effect. To analyze the protein expression of BCL2L10, a cytometry test on HEK293 cells was developed.

For this, HEK293 cells were first transfected with an Myc-tagged BCL2L10 construct “Myc-BCL2L10” and the efficacy of transfection was evaluated using an anti-Myc antibody, as shown in FIG. 19. The expression of the BCL2L10 proteins was confirmed by western blot using an anti-BCL2L10 monoclonal antibody as shown in FIG. 21.

To validate the flow cytometry experiment, a specific siRNA was used, to extinguish the expression of the BCL2L10 gene in HEK293 cells.

Under this condition, neither the expression of the BCL2L10 proteins nor the labeling of BCL2L10 was detected, respectively, by western blot or by flow cytometry, as shown, respectively, in FIGS. 22 and 20. This validates our cytometry experiment based on the detection of BCL2L10 proteins.

### EXAMPLE 2

### The Overexpression of BCL2L10 Involved in the Azacitidine Resistance of SKM1 Cells

Using the assay described in FIGS. 19 to 22, it was established that 73% of SKM1-R cells express the BCL2L10 protein, compared with only 39% of SKM1-S cells, as shown in FIGS. 2 to 4. An increase in the expression of BCL2L10 mRNA and BCL2L10 proteins was also detected in the SKM1-R cells by RT-PCR and by western blot, as shown, respectively, in FIGS. 5 and 6.

To determine whether the overexpression of BCL2L10 is a cause rather than a consequence of azacitidine resistance, the SKM1-S and SKM1-R cells were transfected with a control siRNA or with siRNA directed against one or the other of the BCL2L10 or Bcl-2 proteins, then treated for 24 h with or without azacitidine, before determining cell viability and apoptosis. FIG. 7 shows that the azacitidine led to a loss in cell metabolism in the SKM1-S cells, but not in the SKM1-R cells, as illustrated in FIG. 5. The extinction of the expression of the BCL2L10 gene enables the azacitidine sensitivity of SKM1-R cells to be restored, suggesting an important role of BCL2L10 in the phenomenon of azacitidine resistance. In addition, apoptosis was the main mechanism by which the extinction of the expression of the BCL2L10 gene enabled azacitidine sensitization by increasing the quantity of active caspase-3.

In addition, the labeling of propidium iodide (PI) was detected in the SKM1-R cells treated with a BCL2L10 siRNA, as shown in FIGS. 8 and 9. This effect was specific for BCL2L10 because an siRNA directed against the Bcl-1 protein failed to do so under identical conditions, as shown in FIGS. 8 and 9. Finally, in FIG. 10, it was verified by western blot that the two siRNA's are very effective in blocking the expression of their respective targets. Once combined, our data made it possible to establish that the overexpression of the BCL2L10 protein is responsible for azacitidine resistance in SKM1-R cells.

### EXAMPLE 3

### The Overexpression of BCL2L10 Enables Azacitidine Resistance in Patients with MDS to be Predicted

The expression of BCL2L10 was also analyzed by western blot on samples from patients when the amount of material to be analyzed was sufficient. The results presented in FIGS. 11 to 13 show that the level of BCL2L10 with respect to the level of BCL-2 is variable according to the patients. The ERK protein was used as an internal control for each patient sample. This made it possible to show that the protein expression of BCL2L10 versus ERK is very low in healthy patients, as shown in FIG. 12. Conversely, the expression of the Bcl-2 protein is not significantly different in the 3 groups of patients as illustrated by FIG. 13. The results suggest that the expression of BCL2L10 enables azacitidine resistance to be predicted in patients with MDS.

### EXAMPLE 4

### The Expression of the BCL2L10 Protein is a Biomarker of Azacitidine Resistance in Patients with MDS

The percentage of cells expressing the BCL2L10 protein in the bone marrow of 8 healthy patients, 24 azacitidine-sensitive patients and 8 azacitidine-resistant patients was determined by using the flow cytometry experiment on cohort 1. The clinical characteristics of each patient are provided in tables 1, 2A and 2B below:

TABLE 1
(sensitive patients):

| | | | | Number of | % of | Monitoring |
|---|---|---|---|---|---|---|
| | WHO | IPSS | Karyotype | AZA | BCL2L10- | time |
| Age | classification | category | prognosis | cycles | positive cells | (months) |
| 64 | AML | High | Good | 5 | 30 | 6.5 |
| 77 | RAEB-2 | High | Good | 15 | 5 | 6.1 |
| 80 | AML | High | Good | 11 | 7 | 6.0 |
| 79 | AML | High | Good | 20 | 20 | 5.6 |
| 74 | RAEB-2 | High | Intermediate | 8 | 40 | 7.5 |
| 79 | AML | High | Good | 22 | 16 | 7.5 |
| 67 | RAEB-1 | Int-2 | Good | 6 | 13 | 2.2† |
| 75 | RAEB-1 | Int-2 | Good | 11 | 1 | 4.9 |
| 70 | AML | High | Intermediate | 4 | 4 | 4.7 |
| 77 | RAEB-2 | Int-2 | Good | 4 | 2 | 4.3 |
| 76 | RAEB-1 | Int-2 | Unfavorable | 3 | 0 | 3.7 |
| 68 | RAEB-1 | Int-2 | Intermediate | 14 | 11 | 5.4 |
| 59 | RAEB-2 | High | Intermediate | 13 | 5 | 4.1 |
| 74 | RAEB-2 | High | Intermediate | 11 | 1 | 4.9 |
| 64 | RAEB-2 | High | Unfavorable | 7 | 2 | 3.8 |
| 71 | AML | High | Good | 7 | 28 | 3.6 |
| 80 | AML | High | Good | 14 | 14 | 3.0 |
| 69 | RAEB-2 | High | Intermediate | 17 | 8 | 2.8 |
| 79 | AML | High | Good | 23 | 16 | 2.8 |
| 76 | AML | High | Unfavorable | 7 | 5 | 2.4 |
| 67 | RAEB-2 | High | Good | 12 | 5 | 2.6 |
| 70 | AML | Int-2 | Intermediate | 7 | 31 | 2.0 |
| 74 | RAEB-2 | High | Intermediate | 16 | 40 | 2.0 |

TABLE 2A
(resistant patients):

| | | | | Number of | % of | Monitoring |
|---|---|---|---|---|---|---|
| | WHO | IPSS | Karyotype | AZA | BCL2L10- | time |
| Age | classification | category | prognosis | cycles | positive cells | (months) |
| 69 | RAEB-2 | High | Intermediate | 14 | 64 | 5.7 |
| 69 | AML | High | Unfavorable | 3 | 68 | 3.0† |
| 60 | RAEB-2 | Int-2 | Good | 4 | 72 | 1.5‡ |
| 64 | AML | High | Good | 10 | 93 | 3.8 |
| 64 | RAEB-2 | Int-2 | Good | 19 | 57 | 0.1‡ |
| 76 | RAEB-1 | Int-2 | Unfavorable | 4 | 85 | 0.2† |
| 76 | AML | High | Unfavorable | 4 | 99 | 5.6† |
| 77 | AML | High | Unfavorable | 7 | 95 | 2.3† |

TABLE 2B
(healthy patients):

| | % of BCL2L10- |
|---|---|
| Type of cells | positive cells |
| PNN | 1 |
| CD34 | 18 |
| PBMC | 1 |
| PNN | 2 |
| PBMC | 0 |
| Monocytes | 3 |
| PBMC | 1 |
| Monocytes | 0 |

As is shown in FIG. 14, the average value for the freshly isolated bone marrow samples of healthy patients and azacitidine-sensitive patients is respectively 0%, with values ranging from 0 to 18%, and 8%, with values ranging from 0 to 40%, of cells expressing the BCL2L10 protein, whereas the average value for bone marrow cells from azacitidine-resistant patients is 85%, with values ranging from 57% to 99%, of cells expressing the BCL2L10 protein with a value p of less than 0.0001, as illustrated by FIG. 11. When the samples from 14 patients with low-risk MDS are compared, respectively, with the samples of 21 azacitidine-sensitive patients and 10 azacitidine-resistant patients, all from cohort 2, it is seen that the patients with low-risk MDS have a median of 0% cells expressing BCL2L10, the extremes being 0 and 11%. FIG. 15 also shows that the azacitidine-resistant patients have a much higher percentage of cells expressing the BCL2L10 protein, equal to 33% (p<0.0001), compared with 10% for sensitive patients. In addition, on the basis of the group of patients described in FIG. 8, analysis sub-groups are made. The 10 patients tested who were “at first” refractory to azacitidine have a percentage of cells expressing BCL2L10 equal to 29%, greater than that of the 6 tested patients who were azacitidine-sensitive at diagnosis, which is 10%. These results are shown in FIG. 16 (p=0.023). At the time of relapse, the 4 patients tested who were “at first” azacitidine-sensitive show a percentage of cells expressing the BCL2L10 protein equal to 23%, therefore high compared with the 11 tested azacitidine-sensitive patients under treatment, in whom the percentage of cells expressing the BCL2L10 protein is equal to 14%, as shown in FIG. 17 (p=0.0002).

### EXAMPLE 5

### The Percentage of Cells Expressing the BCL2L10 Protein Predicts the Overall Survival of Patients with MDS and AML

With a reference threshold value, also called “cut-off” value, equal to 50% of cells expressing the BCL2L10 protein, of the total cells of the biological fluid, the test made it possible to obtain excellent positive and negative predictions. In general, the sensitivity and specificity of the test were respectively 80% and 85%.

With a median monitoring time of 4 months, the extremes being 0.1 and 7.5 months, from the data of quantification of BCL2L10, the overall survival (OS) for cohort 1 was significantly better in the sub-groups weakly expressing BCL2L10 than in the sub-groups strongly expressing BCL2L10 (p=0.0016), as shown in FIG. 18a. The graph shown in FIG. 18b shows, over a longer time period (around 15 months, versus around 6 months), the correlation between the percentage of cells expressing BCL2L10 and the overall survival (OS) in patients suffering from MDS or AML treated with azacitidine. This FIG. 18b shows the Kaplan-Meier overall survival curves of the two groups of MDS or AML patients treated with AZA having more or less 50% of cells expressing BCL2L10 in their bone marrow.

An overall survival of 3 months was estimated at 95% for the sub-groups weakly expressing BCL2L10 compared with 51% for the sub-groups strongly expressing BCL2L10. For all of the patients in the sub-group strongly expressing BCL2L10, the disease progressed.

1. In vitro analysis method making it possible to diagnose resistance to an azacitidine treatment in a patient, using the BET2L10 protein contained in a biological fluid sample taken from said patient as well as biological molecules specifically binding the BCL2L10 protein, comprising:
taking a biological fluid sample from a patient;
calculating the percentage of total cells of said biological fluid expressing the BCL2L10 protein;
comparing said calculated percentage with a reference threshold value, said threshold value being between 20 and 60%; and
diagnosing the resistance to an azacitidine treatment in a patient having a higher percentage of cells expressing the BCL2L10 protein in said biological fluid than said reference value.

2. Method according to claim 1, wherein the biological fluid is bone marrow.

3. Method according to claim 1, wherein said reference threshold value is equal to 50%.

4. Method according to claim 1, wherein the measurement of the percentage of cells of the biological fluid expressing the BCL2L10 protein is performed by flow cytometry, by hydrophobic interaction chromatography (HIC), or by quantitative polymerase chain reaction (qPCR), preferably by flow cytometry.

5. Method according to claim 1, wherein the biological molecules specifically binding the BCL2L10 protein are antibodies specific to the BCL2L10 protein.

6. In vitro analysis method making it possible to diagnose resistance to an azacitidine treatment in a patient, by detecting the overexpression of the BCL2L10 gene contained in a biological fluid sample taken from said patient, comprising:
taking a biological fluid sample from a patient;
calculating the percentage of total cells of said biological fluid expressing BCL2L10 by detection of the overexpression of the BCL2L10 gene;
comparing said calculated percentage with a reference threshold, said threshold value being between 20 and 60%; and
diagnosing the resistance to an azacitidine treatment in a patient having a percentage of cells expressing BCL2L10 in said biological fluid greater than said reference threshold.

7. Method according to claim 6, wherein the detection of the overexpression of the BCL2L10 gene is performed by the comparative genomic hybridization CGH method, the flow cytometry method, the ELISA method, the DNA chip method, or quantitative polymerization chain reaction (qPCR).

8. Method according to claim 7, wherein the detection of the overexpression of the BCL2L10 gene is performed by the comparative genomic hybridization CGH method, by the DNA chip method or by quantitative polymerization chain reaction (qPCR).

9. Method according to claim 8, wherein the detection of the overexpression of the BCL2L10 gene is performed by the DNA chip method or by quantitative polymerization chain reaction (qPCR).

10. In vitro analysis kit comprising biological molecules specifically binding the BCL2L10 protein in cells from a biological fluid sample taken from a patient, said kit making it possible to predict resistance to an azacitidine treatment in a patient having a percentage of cells, in said biological fluid expressing the BCL2L10 protein, greater than a reference threshold value of between 20 and 60%.

11. Analysis kit according to claim 10, wherein said biological fluid is bone marrow.

12. In vitro analysis kit comprising at least one reagent selected from the group consisting of:
a pair of primers capable of amplifying a BCL2L10 fragment, and
a probe capable of detecting the presence of BCL2L10,
said kit making it possible to predict resistance to an azacitidine treatment in a patient having a percentage of cells, in said biological fluid expressing BCL2L10, greater than a reference threshold value of between 20 and 60%.

13. Method for monitoring an azacitidine treatment in order to predict relapse, comprising using a kit according to claim 10.

14. Method according to claim 13, which enables said treatment to be adapted according to the response of said patient.

15. Method according to claim 13, wherein said patients have myelodysplastic syndrome and/or acute myeloid leukemia.

16. Method according to claim 2, wherein said reference threshold value is equal to 50%.

17. Method according to claim 16, wherein the measurement of the percentage of cells of the biological fluid expressing the BCL2L10 protein is performed by flow cytometry, by hydrophobic interaction chromatography (HIC), or by quantitative polymerase chain reaction (qPCR), preferably by flow cytometry.

18. Method according to claim 2, wherein the measurement of the percentage of cells of the biological fluid expressing the BCL2L10 protein is performed by flow cytometry, by hydrophobic interaction chromatography (HIC), or by quantitative polymerase chain reaction (qPCR), preferably by flow cytometry.

19. Method according to claim 3, wherein the measurement of the percentage of cells of the biological fluid expressing the BCL2L10 protein is performed by flow cytometry, by hydrophobic interaction chromatography (HIC), or by quantitative polymerase chain reaction (qPCR), preferably by flow cytometry.

20. Method according to claim 2, wherein the biological molecules specifically binding the BCL2L10 protein are antibodies specific to the BCL2L10 protein.
